(19) United States
(12) Patent Application Publication
(10) Pub. No.: US 2003/0153487 A1
(43) Pub. Date: Aug. 14, 2003

(54) Use of npy y1 receptor antagonists in the treatment of inflammatory conditions

(76) Inventors: Patrik Ernfors, Bromma (SE); Philippe Naveilhan, Nantz (FR); Guilherme de Araujo Lucas, Solna (SE); Hassameh Hassani, Sollentuna (SE)

Correspondence Address: YOUNG & THOMPSON, 745 SOUTH 23RD STREET 2ND FLOOR, ARLINGTON, VA, 22202

(21) Appl. No.: 10/257,667

(22) PCT Filed: Apr. 12, 2001
(86) PCT No.: PCT/SE01/00828

(30) Foreign Application Priority Data
Apr. 13, 2000 (SE) 0001373-0

(51) Int. Cl. A61K 38/00

(52) U.S. Cl. 514/2

(57) ABSTRACT

The present invention relates to selective neuropeptide Y (NPY) Y1 receptor antagonists. More closely, it relates to use of, and methods of using, selective NPY Y1 receptor antagonists for the treatment of inflammatory conditions. The invention also relates to use of a NPY Y1 receptor as a drug target in screening procedures to find anti-inflammatory compounds.

### FIELD OF THE INVENTION

The present invention relates to selective neuropeptide Y (NPY) Y1 receptor antagonists. More closely, it relates to use of, and methods of using, NPY Y1 receptor antagonists for the treatment of inflammatory conditions.

### BACKGROUND OF THE INVENTION

Neuropeptide Y (NPY) has a wide range of physiological functions, particularly affecting the cardiovascular system. NPY is also believed to exert antinociceptive actions by inhibiting the release of substance P(SP) and other “pain neurotransmitters” in the dorsal horn of the spinal cord^{1,2,3}. However, the physiological significance and potential therapeutic value remains obscure⁴.

NPY is known to bind with high specificity to several receptor subclasses which have different biological functions. Several pharmacological applications of compounds having NPY receptor agonistic or antagonistic effect have been described.

For example, U.S. Pat. No. 6,017,879 describes template-associated NPY Y2-receptor agonists for treatment of asthma, rhinitis, and bronchitis. An other example is U.S. Pat. No. 5,939,462 describing NPY Y5-receptor antagonist for treatment of obesity.

There is no prior art describing selective NPY receptor agonists having anti-nociciptive action and no prior art describing selective NPY receptor antagonists having anti-inflammatory action.

To have selective drugs is highly desirable in view of less side effects etc.

### SUMMARY OF THE INVENTION

The present invention provides new therapeutic approaches concerning treatment of inflammatory conditions.

In a first aspect, the invention relates to use of, or method of using, a neuropeptide Y Y1 receptor antagonist for preparation of a drug for preventing and/or treating inflammatory conditions.

The NPY Y1 receptor antagonist may be topically, subcutaneously or systemically administered for the treatment of cutaneous inflammation. In an other embodiment, the NPY Y1 receptor antagonist is topically or systemically administered for the treatment of internal inflammation. A preferred use is for treatment of neurogenic inflammation. Another use is for treatment of acute or chronic-persistent inflammation.

When the drug is used systemically in any aspect of the invention, the administration may preferably be orally.

Already available as well as yet unknown compounds having NPY Y1 antagonistic or inhibiting effect can be used for the purposes of the invention. Therefore, in a second aspect, the invention relates to use of the NPY Y1 receptor as a drug target in screening procedures to find antagonists of said receptor, more precisely to find anti-inflammatory compounds which directly or indirectly affect the NPY Y1 receptor in a selective way for treatment of the above described inflammatory conditions. Preferably, high throughput screenings procedures are used to find small organic biocompatible molecules.

To identify a possible physiological role for NPY in pain transduction and to identify the particular receptor subtypes involved, the present inventors generated NPY Y1 receptor null mutant mice (Y1−/−) by homologous recombination techniques.

The present inventors show that Y1−/− mice develop hyperalgesia to acute thermal, cutaneous and visceral chemical pain and exhibit mechanical hypersensitivity. Neuropathic pain is augmented and the mice show a complete absence of the pharmacological analgesic effects of NPY. In the periphery, Y1 receptor activation is sufficient and required for SP release and the subsequent development of neurogenic inflammation and plasma leakage.

The present inventors conclude that the Y1 receptor is required for central physiological and pharmacological NPY-induced analgesia and that its activation is both sufficient and required for the release of SP and initiation of neurogenic inflammation.

### DETAILED DESCRIPTION OF THE INVENTION

Homologous recombination in embryonic stem cells was used to establish mice deficient in the NPY Y1 receptor. The disruption was generated by introducing an internal ribosomal entry site followed by a Tau-LacZ fusion minigene into the second exon of Y1 (FIG. 1a). Southern blot analysis confirmed that the Y1 allele was disrupted and Northern blot analysis showed that instead of the mRNA transcripts encoding Y1, the mutant (Y1^{-/-}) mice produced the expected mRNA encoding β-galactosidase (FIG. 1b-d). As previously described, female Y1^{-/-} mice display a late-onset overweight compared to their littennates⁵ (data not shown). Y1 receptors are abundant in the forebrain while little or nothing is present in the brainstei⁶. Y1 receptors are also highly expressed in dorsal root ganglion neurons in preferentially small and medium size neurons^{6,7}. However, the central termination of Y1 nerve fibers in the dorsal horn, and whether Y1 is expressed in both of the two major cytochemical subpopulations of pain neurons, the SP peptidergic and non-peptidergic pain neurons⁸, is unresolved. β-galactosidase histochemical and immunohistochemical staining of spinal cord sections from Y1^{-/-} mice led to strong staining localised exclusively to the dorsal horn (FIG. 1e and f). Immunohistochemical double staining for β-galactosidase (staining Y1 expressing neurons and fibers) and the lectin IB4 (staining somas and nerve fibers of unmyelinated non-peptidergic sensory nociception neurons⁸) showed a strong staining for Y1 nerve fibers in dorsal horn layer II overlapping with IB4 terminals. A significant portion of the nerve fibers also terminated in layers I, III and IV (FIG. 1f). Y1-positive dorsal horn interneurons were also found (FIG. 1f, arrows). Many Y1 expressing dorsal root ganglion neurons coexpressed SP (FIG. 1h; 38% of Y1 neurons contained SP), however, a large number of Y1 neurons also double stained for IB4 (FIG. 1g; 32%).

A presynaptic block of primary afferent SP release in the spinal cord may participate in central NPY-induced analgesia³. We therefore examined if this primary afferent circuit was intact in the Y1^{-/-} mice. No overt alteration of SP nor NPY immunoreactivity was detected in the spinal cord of Y1^{-/-} mice (data not shown). Furthermore, total SP in spinal cord measured by EIA was 2852±328.9 pg/g tissue and 3919±444.1 pg/g tissue in wild-type and Y1^{-/-} mice, respectively (P>0.05, st udent's t-test). Immunohistochemical detection of the SP receptor revealed similar staining in the somatic and dendritic cell surface of neurons in the superficial spinal cord lamina (I and II) of both wild-type and Y1^{-/-} mice, as in previous results (FIG. 1i and j). SP release leads to SP receptor activation and internalisation. Neurons containing internalised SP receptors were detected in Y1^{-/-} mice following an intraplantar injection of capsaicin (FIG. 1k and 1), indicating that there is no defect of receptor activation per se in the absence of the Y1 receptor. Thus, we conclude that the major neuronal pain circuit suggested to be modulated by NPY is anatomically intact in the Y1^{-/-} mice.

Behavioural acute nociceptive thresholds were markedly affected by an absence of Y1. SP/neurokinin A null mutant mice show a blunted response to painful stimulus only at moderate intensities, whereas response to mildly or intensely painful stimulus is intact¹¹. We therefore tested whether also the NPY Y1 receptor act at specific thresholds in modulating nociception. Withdrawal latency in the hot plate assay was examined at 48, 50, 52, 55 and 58° C. At 48° C. no significant difference was observed (data not shown). The Y1^{-/-} mice showed, however, a profound hyperalgesia and displayed a significantly reduced latency at all temperatures above 48° C. (FIG. 2a). The hot plate test involves supraspinal integration associated with the paw withdrawal. To test whether neuropeptide Y1 receptor could be acting in a spinal circuitry, we characterised these mice in the tail flick assay. Consistent with the hot plate assay, Y1^{-/-} mice showed a markedly reduced latency at all temperatures between 46-54° C. (FIG. 2b). Y1^{-/-} mice also displayed a significant decrease in mechanical threshold indicating mechanical hypersensitivity (FIG. 2c).

We then examined a role for the NPY Y1 receptor in chemical nociception. During the first phase of the formalin assay, which provides a measure of the acute pain mediated by direct chemical activation of C-fibers, the nociceptive behaviour was augmented by 44%, 60% and 46% at 1.2, 2 and 5% formalin injected, respectively, in Y1^{-/-} mice (FIG. 2d). The second phase of nociception was not consistently altered by an absence of the NPY Y1 receptor. In two models of visceral pain, one that is secondary to an inflammatory response (acetic acid) and one that induces immediate pain independent of inflammation (MgSO₄)¹¹, we also found significantly increased pain behaviour in Y1^{-/-} mice (FIG. 2e and f). Combined, the above results indicate that NPY could be acting on Y1 containing polymodal nociceptive afferents (C-fibers). Consistent with our results, these neurons span different modalities, and mediate pain transduction from both visceral and cutaneous tissues.

Stress induces analgesia through endogenous opioid and non-opioid dependent mechanisms¹². We tested whether these pathways interact with NPY-dependent analgesia by letting the mice swim in 10° C. water producing a non-opioid, NADA-dependent analgesia and 33° C. water resulting in opioid-dependent analgesia^{13,14}. After a swim at 10 or 33° C., the development of analgesia assayed in the hot plate assay was similar between wild-type and Y1^{-/-} mice (FIG. 2g). These data suggest that the Y1 receptor is not a critical component in stress-induced analgesia.

The role of NPY in neuropathic pain is incompletely defined and a number of conflicting results with regards to possible NPY receptors involved have been reported^{4,15}. We tested the physiological role of the Y1 receptor in a model of neuropathic pain. A partial sciatic nerve ligation resulted in mechanical allodynia (sensitisation to mechanical stimuli) in wild-type mice (37% and 52% increase in sensitivity at 3 and 14 days after nerve injury, respectively, compared to day 0; FIG. 2h). As indicated before, the basal threshold of mechanical sensitivity was significantly decreased in non-lesioned Y1^{-/-} mice. Despite this, the mechanical allodynia caused by nerve damage was significantly increased in Y1^{-/-} mice compared to wild-type mice (55% and 67% increase in sensitivity at 3 and 14 days after nerve injury, respectively, compared to day 0; P<0.01 for the slopes of the curves between day 0-14 after nerve injury).

Pharmacological NPY-induced analgesia to thermal stimuli following spinal delivery is well documented^{2,16}. To identify the receptor involved in the pharmacological effects of intrathecally administered NPY we injected NPY (10 μg) in the spinal cord of Y1^{-/-} mice and measured heat sensitivity. The anti-nociceptive effect of NPY on the spinal cord was completely abolished in Y1^{-/-} mice (FIG. 2i). Thus, the Y1 receptor is exclusively responsible for the analgesic effects of centrally delivered NPY.

Inflammation is caused by a neurogenic as well as a non-neurogenic component¹⁷. Neurogenic inflammation does not occur in the denervated human skin, and can be prevented by a nerve block in rats^{17,18} and is mediated by a peripheral release of SP/neurokinin A¹¹. We tested whether the Y1 receptor could participate in inflammation. A subcutaneous injection of capsaicin, which induces neurogenic inflammation, led to a marked inflammation seen by an increased paw diameter, plasma extravasation and hyperalgesia in wild-type mice. Unexpectedly, Y1^{-/-} mice displayed no overt or quantitative sign of plasma extravasation, increase in paw diameter and hyperalgesia (FIG. 3a, b, c and d). Neither rectal and paw skin temperature nor heart rate differed between any of the groups before and after capsaicin administration (data not shown). Both wild-type and Y1^{-/-} mice exhibited an increased blood flow in the paw as a response to capsaicin (181.4±31.3% and 245.5±71.4%, respectively) indicating that the Y1 receptor is not influencing capsaicin-induced vasodilation. In contrast to capsaicin, mustard oil induces inflammation that is largely, but not exclusively, dependent on a neurogenic component including the release and proinflammatory effects of SP¹⁹ Y1^{-/-} mice showed markedly reduced plasma extravasation in response to mustard oil compared to wild-type ice. However, since the increase in plasma extravasation was significant (although at a lower level), some components of the effects of mustard oil, likely those of non-neurogenic origin, were intact in Y1^{-/-} mice (FIG. 3h). In contrast to neurogenic inflammation, there was a similar increase of plasma extravasation, paw diameter and sensitisation following non-neurogenic inflammation induced by carrageenan²⁰ in Y1^{-/-} nuce as in wild-type mice (FIG. 3e, f and g).

Since capsaicin and to a large extend mustard oil-induced inflammation depend on the integrity of primary C-fiber afferent release of SP⁸, we determined if Y1 is required prior or after SP release in the sequence of events leading to inflammation by injecting SP in the paw. SP caused a similar inflammatory response in Y1^{-/-} mice as it did in wild-type mice (FIG. 3i).

SP receptor immunoreactivity was intact in the skin of wild-type and Y1^{-/-} mice and was found occasionally in nerve endings in dermis and in scattered cells throughout dermis corresponding to mast cells, similar to wild-type mice (data not shown). Our results are therefore consistent with that Y1 could be required for capsaicin induced SP release. We measured by EIA the quantity of total and released SP in the skin after capsaicin injection in wild-type and Y1^{-/-} mice. Capsaicin caused a marked increase of released SP in the skin of wild-type mice whereas it had no effect on SP release in Y1^{-/-} mice (FIG. 4a). The lack of an increase of released SP was not caused by an overall reduction of SP peripherally because the quantity of total SP was similar in Y1^{-/-} and wild-type mice (2269±524 pg/g tissue and 2520±860 pg/g tissue, respectively). Furthermore, the number of SP immunoreactive nerve fibers in the dermis and epidermis was similar in wild-type and Y1^{-/-} mice (5.7±0.5 and 6.2±0.5 per cm, respectively). In accordance with previous results, capsaicin led to a marked reduction of immunoreactive terminals in the epidermis of wild-type mice (from 3.0±0.3 to 1.8±0.2 per cm; P<0.01, student's t-test) possibly by a loss of immunoreactivity due to increased release. In contrast, Y1^{-/-} mice displayed no reduction in SP immunoreactive terminals (3.1±0.3 and 2.5±0.6 per cm, respectively). These results indicate that the absence of neurogenic inflammation in Y1^{-/-} mice is caused by the requirement of Y1 activation for SP release. The persistent vasodilation in these mice could be caused by a normal release of calcitonin gene related peptide, which induces vasodilation but not extravasation. Furthermore, since close to one order of magnitude less SP is required for vasodilation than for plasma leakage²¹, a small residual SP release in these mice could also cause this phenotype.

We challenged our results in wild-type mice by injecting the Y1 agonist [Leu³¹-Pro³⁴]-NPY and by using a Y1 receptor antagonist. [Leu³¹Pro³⁴]-NPY efficiently caused plasma extravasation in wild-type mice, and consistent with its specificity for the Y1 receptor, no response was seen in Y1^{-/-} mice (FIG. 4b). Administration of the Y1 antagonist BIBP 3226 prior to intr-aplanltar injection of capsaicin markedly reduced plasma extravasation in wild-type mice (FIG. 4c), showing that the results oil the Y1^{-/-} mice are directly caused by the absence of Y1 signalling. We therefore conclude that the NPY Y1 receptor is both required and sufficient to induce neurogenic inflammation by controlling SP release, and that a Y1 antagonist could provide an effective strategy for the treatment of neurogenic inflammatory diseases.

We have shown that the Y1 receptor play an essential anti-nociceptive role during pain transduction in many modalities including thermal, chemical and mechanical from both cutaneous and visceral tissues as well as during neuropathic pain. NPY or another Y1 receptor ligand²² could mediate antinociception by reducing SP and excitatory neurotransmitter release from primary C-fiber afferents^{3,23,24} and/or by inhibiting post synaptically the SP receptor expressing projection neurons of the spinal cord^{25,26}. Consistent with that NPY does not modulate pain transmission only through a presynaptic regulation of SP release, the nociceptive phenotype of the Y1^{-/-} mice does not fully correlate with SP and SP receptor null mutant mice. SP receptor null mutant mice show for instance a reduced stress-induced analgesia²⁷. We also show that Y1 receptor activation is both sufficient and required for neurogenic inflammation. Because mustard oil-induced inflammation occur independent of the vanilloid receptor that is activated by capsaicin¹⁹, our results suggest that activation of the Y1 receptor could be a shared and obligatory component in most, or all, neurogenic inflammatory conditions.

Methods

Y1 gene targeting. Exon 2 of the Y1 gene was partially deleted and replaced by a IRES-tau-lacZ cassette also containing a neomycin-resistance gene driven by the PGK promoter and polyA (ETLN). A 0.7 kb DNA fragment 3′ from the Y1 targeting construct was used as external probe. Homologous recombinant embryonic stem cells clones were injected to generate Y1 mutant 129SVXBalb/c hybrid mice. Mice were analysed by Southern blot and PCR using the primers 5′-ATCAAATTCTGACCGACGAG-3′, 5′-CATGATGTTGATTCGCTTGG-3 and 5′-GCAGCCTCTGTTCCACATACA-3′. Standard procedures were used for Northern blot analysis and 60 μg/sample of total RNA from adult brain was analysed.

Physiological studies. Heart rate and body temperature was measured as previously described²⁸. Skin blood flow and temperature was monitored with a thermal probe (Physitemp bat-12) subcutaneously inserted in the plantar region of the paw and a Laser doppler flowmeter probe applied to the plantar surface (Penmided PF2B) was used to measure basal and capsaicin-evoked changes. Student t-test was used and differences were considered significant at P<0.05.

Behavioural studies. Between 6-13 adult male F2-3 129SV×Balb/c mice of each genotype were used for all studies. Thermal¹¹, mechanical¹¹, chemical¹¹. visceral¹¹ sensitivity, stress-induced analgesia²⁷ and neuropathic pain^{29,30} was assessed as previously. Tail-flick latency (by directing a concentrated light beam to the tail of the mouse) was monitored before and after intrathecal injection of 10 μg NPY. For evans blue plasma extravasatioin the following were used: capsaicin, 3 μg in 10 μl (Sigma; dissolved in 5% ethanol, 5% Tween-80 and 90% saline), 1% carrageenan (Sigma; dissolved in saline), SP, 50 pmol/paw (Sigma dissolved in saline), 5% mustard oil (Fluka; dissolved in mineral oil), NPY Y1 receptor agonist [Leu³¹-Pro³⁴]-NPY, 10 μg/paw (Calbiochem; dissolved in saline and 5% acetic acid) and NPY Y1 receptor selective antagonist BIBP 3226, 10 mg/kg in 10 ml/kg (American Peptide; dissolved in saline and 5% acetic acid) Briefly, mice were anaesthetised and injected intravenously with Evan's Blue (50 mg/kg) into the jugular vein. Agents mentioned above were injected into one paw of the animal except for Y1 receptor selective anatagonist, BIBP 3226, which was injected intravenously 10 min prior to injection into the paw. The other paw was injected with vehicle. After 30 min the plantar skin of the paw was removed, dried off excess liquid, weighed and incubated in formamide for 24 h at 56° C. Extravasated evans blue was measured by spectrophotometer at 620 nm. Mechanical sensitivity was determined before, 30 min and 3 h after capsaicin and carrageenan administration, respectively. Carrageenan inflammation was induced similarly but extravasation was measured after 4 hours. The paw diameter was measured before and after capsaicin, carrageenan or vehicle administration using a spring-loaded calliper.

Immunohistochemistry. Wild-type and Y1^{-/-} mice were perfused with 4% paraformaldehyde (for SP receptor immunohistochemistry, mice were perfused with 4% paraformaldehyde and 12.5% picric acid 10 min after capsaicin injection into hindpaw) and the spinal cord and dorsal root ganglia were sectioned coronally (15 μL in thickness). Capsaicin was injected intradermally into dorsal skin of mice. After 10 min the skin was removed, postfixed and sectioned as above. Immunohistochemistry was performed as previously²⁸ using α-β-galactosidase (1:200 dilution, ICN/Cappel) rabbit ctSP (1:5000 dilution, Chemicon), guinea pig α-SP (1:200 dilution, Peninsula Lab.), rhodamine-conjugated bandeiraea simplicifolia lectin I (Isolectin B4; 1:100 dilution, Vector), α-NPY (1:200 dilution, Peninsula Lab.), and rhiodamine or FITC-conjugated secondary antisera (Jackson). For SP receptor immunohistochemistry sections were incubated 30 min in PBS, 50% methanol and 0.6% H₂O₂ prior incubation in 10% goat sermum. The antiserum (Chemicon 1:2000) was used in the fluorescein TSA fluorescence system (NEN). β-galactosidase histochemical staining was performed as previously^{28.}

EIA. Capsaicin or saline was injected into the paw of WT or Y1^{-/-} mice. After 10 min, the paw was removed and the skin was cut open and washed in PBS and 0.1% BSA for 10 min. The skin was then dried, weighed, transferred to a new container and frozen. The liquid was centrifuged at 4000 rpm for 15 min. Supernatant was transferred to a new tube, weighed and frozen. The lumbar part of spinal cord was removed, weighed and frozen. The samples were then assayed for SP according to the manufacturer's instructions using SP high sensitivity EIA kit (Peninsula Lab.).

### FIGURE LEGENDS

FIG. 1. Targeted mutagenesis of the Y1 receptor and expression analysis of Y1 and SP receptors. a, Y1 gene-targeting. Top, targeting vector (Y1 coding exons=black boxes). The disrupting cassette is indicated. Bottom, restriction map of the resulting targeted allele (B-BamHI Sp-SpeI; E-EcoRI; P-PacI; Pr, probe used in the Southern blots). b, Southern blot analysis of ES cells. c, PCR genotyping of wild-type, Y1^{+/-} and Y1^{-/-} mice. d, Northern blot analysis of total brain RNA of Y1⁺⁺ and Y1^{-/-} mice Losing a Y1 probe (Y1 Pr) or LacZ probe (LacZ Pr). Probes used are underlined in red in (a). e, A transverse section from the spinal cord lumbar enlargement of Y1^{-/-} mice histochemically stained for β-galactosidase. f, Immunohistochemical staining of Y1^{-/-} mice for β-galactosidase-positive nerve terminals and neurons (arrows) in the spinal cord dorsal horn (green) and the lectin IB4 (red, layer IIinner). g, Double staining of L4 dorsal root ganglion for β-galactosidase (green) and IB4 (red). h, Double staining of L4 dorsal root ganglion for β-galactosidase (green, single stained neurons=arrows) and SP (red). Double stained neurons are shown by arrowheads. i, SP receptor distribution in the dorsal horn of wild-type mice. j, SP receptor distribution in dorsal horn of Y1^{-/-} mice. k, SP receptor staining in lamina I of the contralateral vehicle injected side of Y1^{-/-} mice. l, Loss of cell surface and increase of intracellular SP receptor immunoreactivity in lamina I ten minutes after capsaicin injection into the hindpaw of Y1^{-/-} mice. Scale bar in (e) is 300 μm, in (f), (i) and (j) 80 μm, in (g) and (h) 30 μm, in (k) and (l) 20 μm.

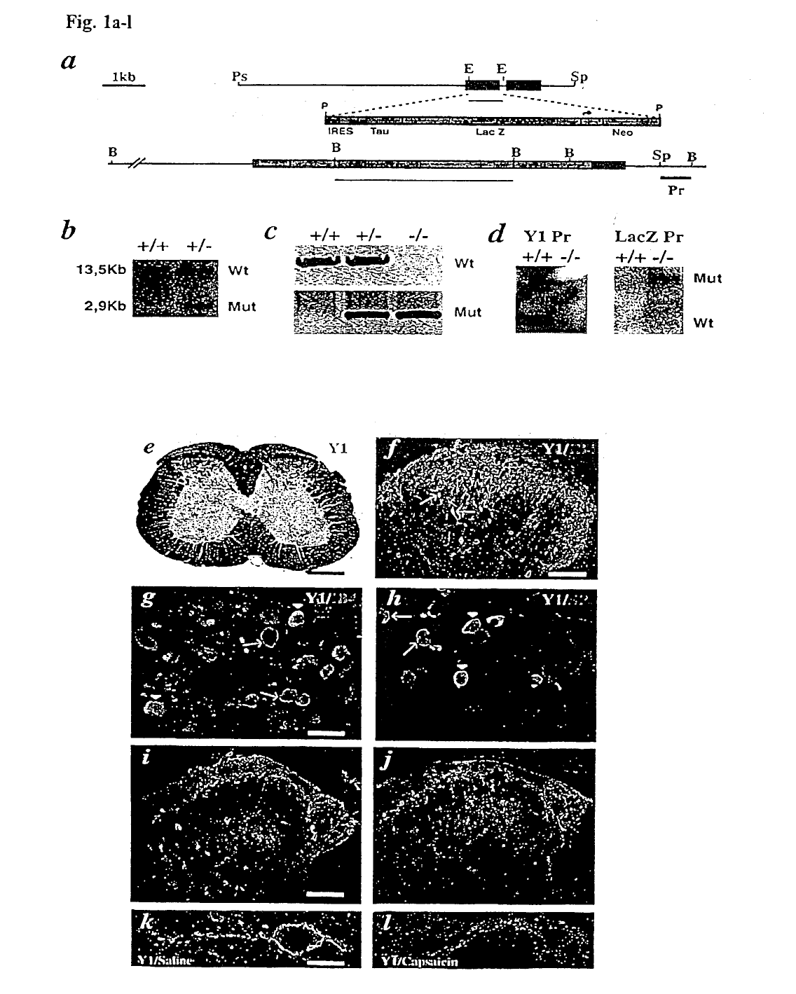

FIG. 2. Cutaneous and visceral nociception of wild-type (black bars) and Y1^{-/-} (white bars) mice in the hot-plate, tail-flick, formalin, acetic acid, MgSO₄, von Frey hair and in neuropathic pain assays as well as in stress and NPY produced analgesia. a, Latency to shaking of hind-paw or jumping. b Tail-flick latency. c, Mechanical threshold assayed by von Frey hairs. d, Measurement of the number of events (lifting, shaking, licking and biting of the injected paw) in the formalin assay. The numbers on the X-axis indicate the concentration in percent of formalin administered subcutaneously. e and f, Visceral pain response (abdominal stretching) produced by intraperitoneal injection of diluted acetic acid (e), or MgSO₄ (t). g, stress-induced analgesia in the hot plate assay. l, Development of mechanical allodynia of wild-type and Y1^{-/-} mice in a chronic pain model. i, Analgesic response to tail-flick following an intrathecal injection of NPY. Data are presented as % analgesia. All data are mean±SEM and statistical analysis was performed by unpaired student's t-test (a-g and i) or two-tailed Maru Whitney U-test (h). *, P<0.05; ** P<0.01; ***, P<0.001.

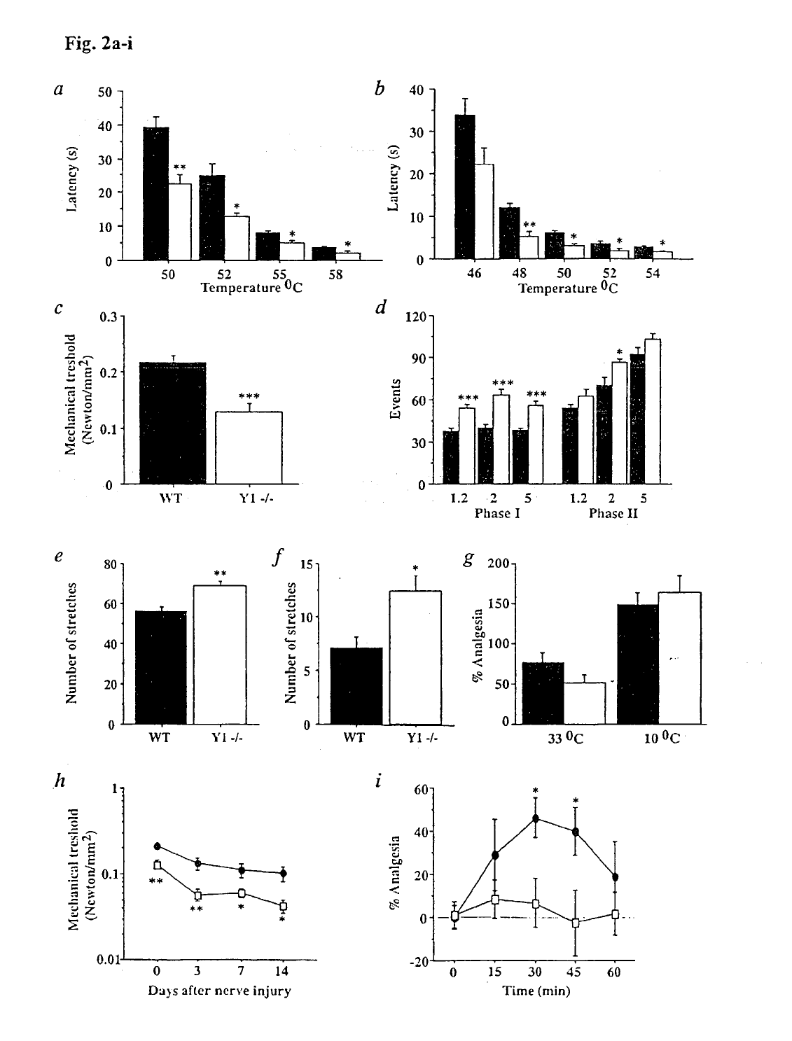

FIG. 3. Neurogenic and non-neurogenic inflammation in wild-type and Y1^{-/-} mice. a, Paws of wild-type and Y1^{-/-} mice 30 min after injection of capsaicin (neurogenic inflammation) or vehicle. b, Quantification of evans blue extravasation after capsaicin or vehicle injection. c, Percentage of paw diameter increase of vehicle and capsaicin injected paws. d, Mechanical sensitisation before and after capsaicin-induced inflammation. e and f, Evans blue extravasation (e) and paw diameter (f) 4 hours after carrageenan (non-neurogenic) induced inflammation in the wild-type and Y1^{-/-} mice as indicated. g, Mechanical sensitisation 3 h after carrageenan induced inflammation. In, Quantification of evans blue extravasation of the paws 30 min after mustard oil administration. i, Quantification of evans blue extravasation 30 min after vehicle or SP administration. In all experiments open bars are vehicle control side and black bars the experimental side. All data are mean±SEM. Statistical analysis was performed by unpaired student's t-test. *, P<0.05; **, P<0.01; ***, P<0.001.

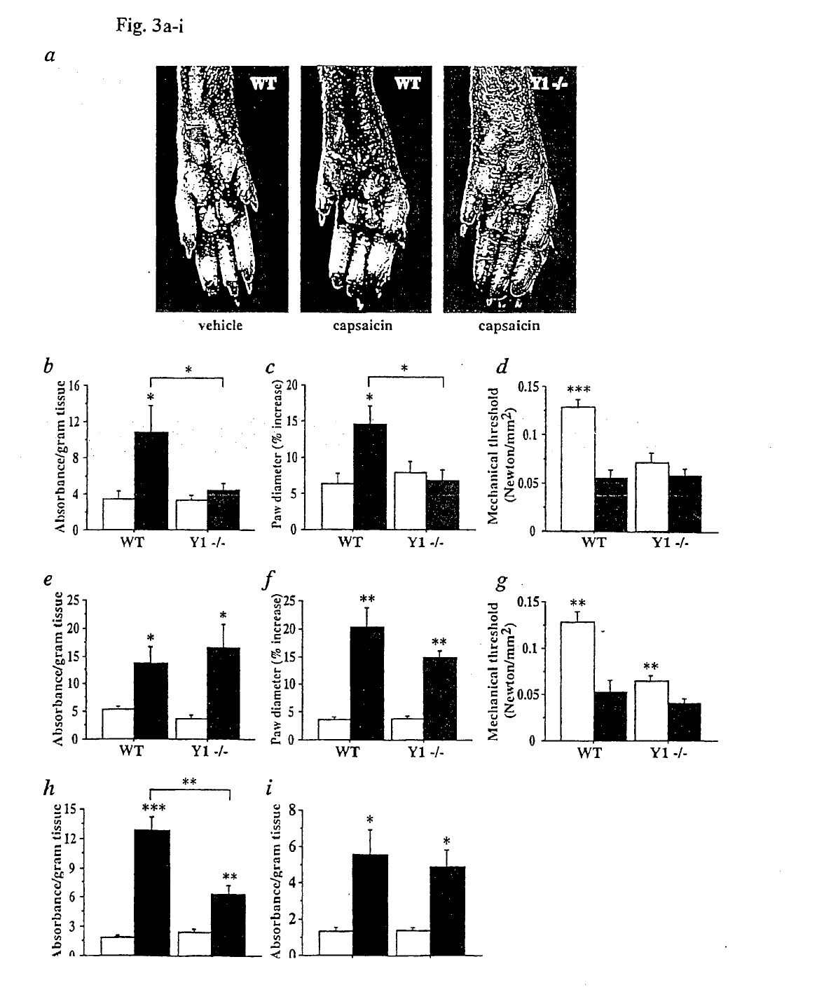

FIG. 4. Measurement of SP release by capsaicin administration in the skin by EIA and effects of Y1 agonist and antagonist in inflammation-induced plasma extravasation. a, Released SP in vehicle and capsaicin injected skin. b, Evans blue extravasation 30 min after NPY Y1 receptor agonist [Leu³¹-Pro³⁴]-NPY or vehicle injection intraplantarly. c, Capsaicin-induced evans blue extravasation in wild-type mice in the presence or absence of NPY Y1 receptor selective antagonist BIBP 3226. In all experiments open bars are the vehicle control side and black bars the experimental side. All data are mean±SEM and statistical analysis was performed by unpaired student's t-test. *, P<0 05; **,P<0.01; ***, P<0.001.

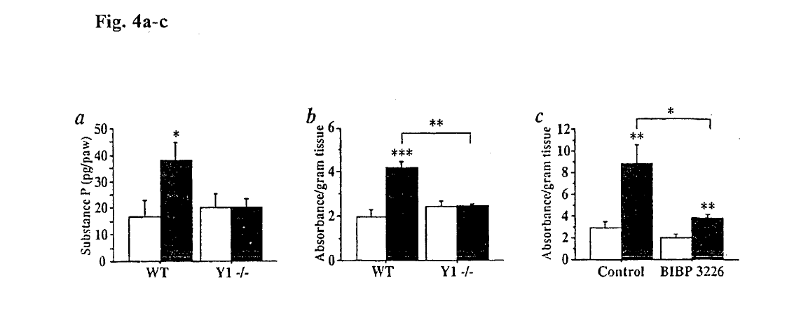

### REFERENCES

1. Munglani, R., Hudspith, M. J. & Hunt, S. P. The therapeutic potential of neuropeptide Y. Analgesic, anxiolytic and antihypertensive. Drugs 52, 371-389 (1996).

2. Hua, X Y. et al. The antinociceptive effects of spinally administered neuropeptide Y in the rat: systematic studies on structure-activity relationship. J Pharmacol Exp Ther 258, 243-248 (1991).

3. Duggan, A. W., Hope, P. J. & Lang, C. W. Microinjection of neuropeptide Y into the superficial dorsal horn reduces stimulus-evoked release of immunoreactive substance P in the anaesthetized cat. Neuroscience 44, 733-740 (1991).

4. Hudspith, M. J. & Munglani, R. Neuropeptide Y: friend or foe. Eur J Pain 3, 3-6 (1999).

5. Pedrazzini, T. et al. Cardiovascular response, feeding behavior and locomotor activity in mice lacking the NPY Y1 receptor. Nat Med 4, 722-726 (1998).

6. Naveilhan, P., Neveu, I., Arenas, E. & Ernfors, P. Complementary and overlapping expression of Y1, Y2 and Y5 receptors in the developing and adult mouse nervous system. Neuroscience 87, 289-302 (1998).

7. Zhang, X. et al. Localization of neuropeptide Y Y1 receptors in the rat nervous system with special reference to somatic receptors on small dorsal root ganglion neurons. Proc Natl Acad Sci USA 91, 11738-11742 (1994).

8. Snider. W. D. & McMahon, S. B. Tackling pain at the source: new ideas about nociceptors. Neuron 20, 629-632 (1998).

9. Allen, B. J. et al. Primary afferent fibers that contribute to increased substance P receptor internalization in the spinal cord after injury. J Neurophysiol 81, 1379-1390 (1999).

10. Mantyh, P. W. et al. Receptor endocytosis and dendrite reshaping in spinal neurons after somatosensory stimulation. Science 268, 1629-1632 (1995).

11. Cao, Y. Q. et al. Primary afferent tachykinins are required to experience moderate to intense pain. Nature 392, 390-394 (1998).

12. Watkins, L. R. & Mayer, D. J. Multiple endogenous opiate and non-opiate analgesia systems: evidence of their existence and clinical implications Ann NY Acad Sci 467, 273299 (1986).

13. Rubinstein, M. et al. Absence of opioid stress-induced analgesia in mice lacking beta-endorphin by site-directed mutagenesis. Proc Natl Acid Sci USA 93, 3995-4000(1996).

14. Marek, P., Mogil, J. S., Sternberg, W. F., Panocka, I. & Liebeskind, J. C. N-methyl-D-aspartic acid (NMDA) receptor antagonist MK-801 blocks non-opioid stress-induced analgesia. Il. Comparison across three swim-stress paradigms in selectively bred mice. Brain Res 578, 197-203 (1992).

15. Zhang, X., Wiesenfeld-Hallin, Z. & Hokfelt, T. Effect of peripheral axotomy on expression of neuropeptide Y receptor mRNA in rat lumbar dorsal root ganglia. Eur J Neurosci 6, 43-57 (1994).

16. Xu, X. J., Hao, J. X., Hokfelt, T. & Wiesenfeld-Hallin, Z. The effects of intrathecal neuropeptide Y on the spinal nociceptive flexor reflex in rats with intact sciatic nerves and after peripheral axotomy. Neurosciece 63, 817-826 (1994).

17. Jancso, N., Jancso-Gabor. A. & Szolcsanyi, J. Direct evidence for neurogenic inflammation and its prevention by denervation and by pretreatment with capsaicin. Br J Pharmacol 31, 138-151 (1967).

18. Jancso, N., Jancso-Gabor, A. & Szolcsanyi, J. The role of sensory nerve endings in neurooenic inflammation induced in human skin and in the eye and paw of the rat. Br J Pharmacol 33, 32-41 (1968).

19. Inoue, H., Asaka, T., Nagata, N. & Koshihara, Y. Mechanism of mustard oil-induced skin inflammation in mice. Eur J Pharamacol 333, 231-240 (1997).

20. Semos, M. L. & Headley, P. M. The role of nitric oxide in spinal nociceptive reflexes in rats with neurogenic and non-neurogenic peripheral inflammation. Neuropharmacology 33, 1487-1497 (1994).

21. Lembeck, F. & Holzer, P. Substance P as neurogenic mediator of antidromic vasodilation and neurogenic plasma extravasation. Naunyn Schmiedelbergs Arch Pharmacol. 310, 175-183 (1979).

22. Shi, T. J., Zhang, X., Berg, O. G., Erickson, J. C., Palmiter, R. D. & Hokflt, T. Effect of peripheral axotomy on dorsal root ganglion neuron phenotype and autotomy behaviour in neuropeptide Y-deficient mice. Regul Pept 25, 161-173 (1998).

23. Wilcox G. L. Excitatory neurotransmitters and pain (ed. Bond M. R., Charlton J. R., &, Woolf C. J.) 97-117 (Elsevier Science Publishers BV, New York, 1991).

24. Walker, M. W., Ewald, D. A., Perney, T. N4. & Miller. R. J. Neuropeptide Y modulates neurotransmitter release and Ca2+ currents in rat sensory neurons. J Neurosci 8, 2438-2446 (1988).

25. Polgar, E., Shehab, S. A., Watt, C. & Todd, A. J. GABAergic neurons that contain neuropeptide Y selectively target cells with the neurokinin I receptor in laminae III and IV of the rat spinal cord. J Neurosci 19, 2637-2646 (1999).

26. Parker, D. et al. Co-localized neuropeptide Y and GABA have complementary presynaptic effects on sensory synaptic transmission. Eur J Neurosci 10, 2856-2870 (1998).

27. De Felipe, C. et al. Altered nociception, analgesia and aggression in mice lacking the receptor for substance P. Nature 392, 394-397 (1998).

28. Naveilhan, P. et al. Normal feeding behavior, body weight and leptin response require the neuropeptide Y Y2 receptor. Nat Med 5, 1188-1193 (1999).

29. Seltzer, Z., Dubner, R. & Shir, Y. A novel behavioral model of neuropathic pain disorders produced in rats by partial sciatic nerve injury. Pain 43, 205-218 (1990).

30. Malmberg, A. B. & Basbaum, A. I. Partial sciatic nerve injury in the mouse as a model of neuropathic pain: behavioral and neuroanatomical correlates. Pain 76, 215-222 (1998).

1. Use of a selective neuropeptide Y Y1 receptor antagonist for preparation of a drug for preventing and/or treating inflammatory conditions.

2. Use according to claim 1, wherein the NPY Y1 receptor antagonist is topically, subcutaneously or systemically administered for the treatment of cutaneous inflammation.

3. Use according to claim 1, wherein the NPY Y1 receptor antagonist is used topically or systemically for the treatment of internal inflammation.

4. Use according to claim 1, wherein the NPY Y1 receptor antagonist is used for treatment of neurogenic inflammation.

5. Use according to one or more of claims 1-4, wherein the NPY Y1 receptor is used for treatment of acute and chronic/persistent inflammation.

6. Use of a NPY Y1 receptor as a drug target in screening procedures to find anti-inflammatory compounds.
